(19) United States
(12) Plant Patent Application Publication
(10) Pub. No.: US 2015/0359151 P1
(43) Pub. Date: Dec. 10, 2015

(54) Coast redwood with periclinal chimeric albinism named 'Axillares Albus'

(50) ;

(71) Applicants: Thomas Santos Stapleton, Volcano, CA (US); Dale Frederick Holderman, Santa Cruz, CA (US)

(72) Inventors: Thomas Santos Stapleton, Volcano, CA (US); Dale Frederick Holderman, Santa Cruz, CA (US)

(21) Appl. No.: 14/120,624

(22) Filed: Jun. 10, 2014

(51) Int. Cl. A01H 7/00

(52) U.S. Cl. PLT/213

(57) ABSTRACT

‘Axillares Albus’ is a new and distinct variety of albino coast redwood characterized by a non-grafted periclinal chimera exhibiting stable albino growth from inside the apical meristem dome. The new variety contains lateral, adventitious, accessory, and dorsoventral axillary buds on the abaxial side of the ramus, with phenotypic expression of color ranging from green and albino, and chimeric or non-chimeric variegation. Further, the branches demonstrate horizontal to weeping-like habit, and moderate to fast growth compared to other common redwoods.

### LATIN NAME OF THE GENUS AND SPECIES OF THE PLANT CLAIMED

The Coast Redwood variety of this invention is botanically identified as Sequoia sempervirens.

### VARIETY DENOMINATION

The variety denomination is ‘Axillares Albus’.

### BACKGROUND OF THE INVENTION

The present invention relates to a new and distinct plant variety of Sequoia sempervirens, more commonly known as Coast Redwood, having naturally-occurring chimeric characteristics resulting in albinism vegetation.

Specifically, ‘Axillares Albus’ is a periclinal chimera exhibiting stable albino growth inside the apical meristem dome. Tests conducted by the first-named inventor reveal that the present invention has a survival tolerance of up to 65 percent albinism. The buds of the present invention contain several forms of naturally-occurring chimera, including: periclinal, mericlinal, and sectorial. The branches demonstrate color variation, including: green and albino, and chimeric, and non-chimeric variegation; and the branches exhibit a horizontal to pendulous growth habit. Additionally, under the right conditions, the present invention yields high propagation levels via stem and leaf cuttings.

The new variety originated as a result of an ongoing breeding program in Santa Cruz, Calif. The seedling of the present invention was grown from a controlled cross conducted in 1976 where an unnamed, unpatented albino Sequoia sempervirens providing the male pollen, and the conelets of an unnamed, unpatented green Sequoia sempervirens were pollinated. After repeated experimental propagation efforts, asexually-reproduced leaf and stem cuttings took root in a greenhouse laboratory setting in Volcano, Calif. in 2012. These cuttings yielded the stable periclinal chimeric albinism growth characteristic of the present invention.

Coast redwood trees (herein referred to as “redwood”) are well known in the industry for their disease and insect resistance, fast growth habit, fire tolerance, and for supporting wildlife habitat. Redwoods are also noted for their height and longevity. These characteristics have led to an increase of the use of this plant as an ornamental feature in landscaped gardens and re-forestation projects. Congruent with similar Coast Redwoods, the present invention is anticipated to reach a height of 18 to 36 meters, and have a lifespan capacity of 200 to 1000 years.

Albinism in redwoods is a genetic mutation presenting as chlorophyll deficit in the plant's needles and stems. As chlorophyll is instrumental to glucose production and storage, albinism prevents a plant from providing food for itself; therefore, survival of albino growth is depended upon parasitic-type growth on the non-albino portions of the redwood. Albino redwoods in the wild are typically found in two forms: aerial and basal. Aerial albinos consist of a mutated branch were the foliage grows white. Basal albinos consist of entirely white basal sprouts growing off an otherwise healthy green redwood. A chimeric redwood is a single plant organism with two or more different genotypes. The normal green genotype acts as a surrogate to support the growth and survival of the albino mutation. It is not a symbiotic relationship between two separate plants. Further, due to this dependency and lack of chlorophyll, pure albino redwoods are unable to be reproduced vegetatively.

White color variation is subject to environmental conditions, particularly light exposure. For example, when grown in direct sunlight conditions, albino redwoods may turn ivory, cream, or light yellow in color. In contrast, when growing under the canopy of surrounding tree branches, albino redwoods are white in color. Excessive heat and low humidity may also result in die-back of the albino portions of the redwood. Additionally, the variation and distribution of white coloration is influenced by the phenotypic expression of three different types of chimeric growth: periclinal, sectorial, and mericlinal.

Chimerism in plants is typically achieved through grafting and controlled gamma ray irradiation of seeds. One example of a grafted chimera plant is the thornless rose. A second example of irradiated seeds producing chimeric growth is the African violet plant. The present invention, in contrast, is a naturally-occurring chimeric mutation observed through variegated apical stems.

### DEFINITIONS

In order to provide a clear and consistent understanding of the specification, the following definitions are provided:

Albino. “Albino” refers to a white color variation ranging from ivory white to pale, yellow-green white, and is a result of a genetic mutation inhibiting chlorophyll production.

Chimera. “Chimera” refers to the existence of more than one genotype present in one plant. The chimeric phenotype is separated into three different categories based on the location and relative proportion of mutated to non-mutated cells in the apical meristem. These categories are mericlinal, periclinal, and sectorial.

Chimeric variegation. “Chimeric variegation” refers to a pronounced delineation of color with both green and white pigment in the meristems and needles.

Mericlinal chimera. “Mericlinal chimera” refers to a phenotypic expression in which only a small portion of the plant structure (stems, branches, & leaves) demonstrates chimeric albinism. This type of chimera is known in the industry to be unstable.

Non-chimeric variegation. “Non-chimeric variegation” refers to a: partial lack of chlorophyll (and therefore green pigment) in plant cells and tissues where it is normally expected to be present. The pattern of variegation is unorganized and is differentiated at the cellular level between green and white. The Variegation expression is mosaic in appearance.

Periclinal chimerism. “Periclinal chimerism” refers to a stable chimeric variegated mutation expressing albinism across the meristem dome. This leads to subsequent cell division of mutated and non-mutated cells within the meristem giving rise to a stable continuation of growth for both genotypes.

Sectorial chimera. “Sectorial chimera” refers to growth where mutated cells affect large sections of the apical meristem. Mutated tissue can extend through all cell layers within the meristematic tissue. The delineation line between both genotypes is usually vertical in arrangement on the meristem. This type of chimerism is known in the industry to be unstable.

### BRIEF SUMMARY OF THE INVENTION

The following traits represent the characteristics of the new redwood variety ‘Axillares Albus’. These traits in combination distinguish this variety from all other commercial varieties known to the inventors.
1. A non-grafted, periclinal chimera exhibiting stable albino growth from inside the apical meristem dome;
2. Lateral, adventitious, and dorsoventral axillary buds on the abaxial side of the ramus with color expression ranging from green and albino, and chimera or non-chimeric variegation;
3. Horizontal to weeping-like branches; and
4. Moderate to fast growth when compared to other common redwoods.

The initial cross taking place in 1976 under the direction of the second-named inventor, combined the premature cones on the green redwood (Parent Two) with pollen collected from the albino redwood (Parent One). The conelets were then sealed with a plastic bag to prevent open pollination. Redwood seeds take a year to mature, so in late 1977, of the thousands of seeds present, 360 were randomly selected from the ripe cones. Within a couple of weeks, several tiny stems and cotyledons emerged displaying differences in coloration. The present invention is derived from one of the 161 surviving seedlings of this experiment. Further background on the original cross are detailed in the book entitled The White Redwoods: Ghosts of the Forest (Davis, D. & Holderman, D. 1980, Naturegraph Publishers. California. Pages 33-36).

Presently, the invention demonstrates a slow-growing, bush-like growth habit. It is approximately 1.52 m tall with a flat top and a stem nearly 10.2 cm in diameter. The limb spread is about 4.6 m in diameter. The foliage exhibiting periclinal chimera is relatively sparse and open, while sections with only albino and green foliage appear more dense and compact. Ten percent of the present invention's branches exhibit periclinal chimeric growth with albino axillary bud growth. Propagated cuttings from the present invention already exhibit a faster growth rate than the present invention. The cuttings taken from the present invention are expected to present a tree habit because the cuttings are propagated as leaf cuttings, where it is speculated that an absence of growth regulator hormone will not stifle growth.

Experimental propagation of the present invention underwent several propagation efforts and experienced several challenges in producing vigorous specimens. Then, in the spring of 2012, the first-named inventor selected seven softwood cuttings off the present invention. The cuttings were transported to a greenhouse located in Volcano, Calif. The cuttings were divided into stem and leaf cuttings, and dipped into a rooting solution consisting of 2500 PPM of IBA for approximately 10 seconds. Following this step the stem and leaf cuttings were planted in gallon-sized pots and treated with a fungicide product. Between the Fall of 2012 through the Winter of 2012-2013, the cuttings were misted and given water at regular intervals. The cuttings were also provided supplemental lighting 24 hours a day. By the end of August 2013, the cuttings demonstrated increased albino growth from dorsoventral axillary buds the abaxial side of the ramus.

The present invention has been asexually propagated in a controlled nursery environment through vegetative, leaf and stem cuttings under the direction of the first named inventor. The cuttings of the new variety, ‘Axillares Albus’, have demonstrated that the combination of characteristics disclosed are stable and firmly fixed and are retained true-to-type through the periclinal chimera genotypes. It is important to note that the invention can exhibit mericlinal and sectorial growth through the periclinal phenotype and therefore this growth is claimed within this invention. It is known in the literature that periclinal chimerism in plants is considered stable and as such, is readily available in commercial markets (Lineberger, R. No date. Origin, Development, and Propagation of Chimeras. Texas A & M University. Retrieved from http://aggie-horticulture.tamu.edu/tisscult/chimeras/chimeralec/chimeras.html on Jan. 20, 2014).

During propagation experimentation, one cutting was selected for a fertilizer test and the conclusion of the test elucidates that a slow release fertilizer works best with ‘Axillares Albus’ and quick release fertilizers should be avoided.

Propagation experiments reveal that coloration and growth rates will vary somewhat between cuttings depending on whether the cutting is taken from the stem or the leaf. Table 1 illustrates the variations of the seven original cuttings at approximately fourteen months of age.

TABLE 1

| | Type of | | New vertical | | % with | | | Number of | |
|---|---|---|---|---|---|---|---|---|---|
| Cutting | Cutting | Height | growth | | albinism | Longest branch | | albino buds | Notes: |
| #1 | Leaf | 99.7 cm | 92.1 | cm | 40% | 48.9 | | 31 | N/A |
| #2 | Leaf | 84.5 cm | 77.6 | cm | 35% | 38.1 | cm | 24 | N/A |
| #3 | Stem | 78.7 cm | 71.1 | cm | 20% | 33.0 | cm | 15 | N/A |
| #4 | Stem | 36.8 cm | 17.1 | cm | 55% | 21.6 | | 23 | N/A |
| #5 | Stem | 20.3 cm | 7.6 | cm | 50% | 31.8 | cm | 15 | N/A |
| #6 | Leaf | 35.6 cm | 29.6 | cm | 0% | 22.2 | cm | 0 | N/A |

| | | | | | | | |
|---|---|---|---|---|---|---|---|
| #7 | Stem | N/A | N/A | N/A | N/A | N/A | Failed to |
| | | | | | | | root |

It is known in the literature that conifers (redwoods) may experience delayed axillary bud emergence from the meristem (for example, see Fink, S. 1984. Some Cases of Delayed or Induced Development of Axillary Buds From Persisting Detached Meristems in Conifers. Amer. J. Bot. 71(1) Pages 44-51). This natural delay has been observed in the present invention, wherein white axillary buds emerged from established green buds at least one year after the initial meristem growth even though the cuttings were grown under the same conditions. For example, between the age of seven and fourteen months, Cutting Number 2 increased its albinism from 10 percent to 35 percent, Cutting Number 3, from 5 percent to 20 percent, and Cutting Number 4, from 35 percent to 55 percent.

Plant Breeder's Rights for this variety have not been applied for and ‘Axillares Albus’ has not been offered for sale more than a year before the filing date of this application. Since the original cross, cuttings of ‘Axillares Albus’ have undergone experimental use to solve prior propagation and the present invention has not been publicly available during this time.

Plants of the present invention have not been observed under all possible environmental and cultural conditions. The phenotype may vary somewhat with variations in environmental conditions without, however, any variance in genotypes. For example, phenotypic expression may vary somewhat with fluctuations in temperature, light intensity and soil chemistry. Observations and characteristic data disclosed herein were collected in the spring of 2014 in Volcano, Calif.

### BRIEF DESCRIPTION OF THE PHOTOGRAPH(S)

The accompanying colored photographs illustrate the overall appearance of the new and distinct albino redwood plant with periclinal chimerism showing the colors as true as it is reasonably possible to obtain in colored reproductions of conventional photography. Two typical specimens of the present invention are included to demonstrate color variation on the leaves and stems produced by the chimeric and non-chimeric genotypes.

FIG. 1 is a close-up image of propagated Leaf Cutting Number 2, demonstrating the albinism of the apical meristem dome and the dorsoventral axillary buds on the abaxial side of the ramus with green and white variegation. The photograph was taken in May 2014 in a greenhouse setting under a combination of natural and artificial lighting.

FIG. 2 (of Leaf Cutting Number 2) best demonstrates the characteristics of the propagated invention's horizontal to weeping-like habit. Leaf Cutting Number 2 is the cutting used for the detailed description. The photograph was taken in May 2014 in a greenhouse setting under artificial lighting.

FIG. 3 is an image of a sibling to the present invention and was taken in 1977 in Santa Cruz, Calif. It demonstrates the same likeness to what the invention looked like at the time of germination. The cotyledons exhibit chimeric variegated growth similar to the present invention.

FIG. 4 demonstrates the bush-like growth habit and white and green coloration. FIG. 4, taken in the Summer of 2012, is of the present invention at approximately 36 years of age where it is growing outdoors in a cultivated area in Santa Cruz, Calif.

FIG. 5 demonstrates the chimeric growth of the dorsoventral axillary buds on the abaxial side of the ramus where softwood cuttings were taken from the present invention for propagation. FIG. 5, taken in the Summer of 2012, is of the present invention at approximately 36 years of age where it is growing outdoors in a cultivated area in Santa Cruz, Calif.

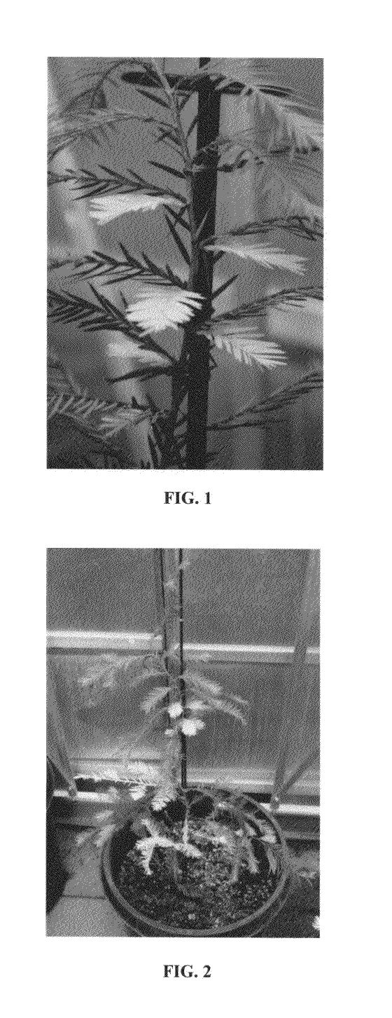

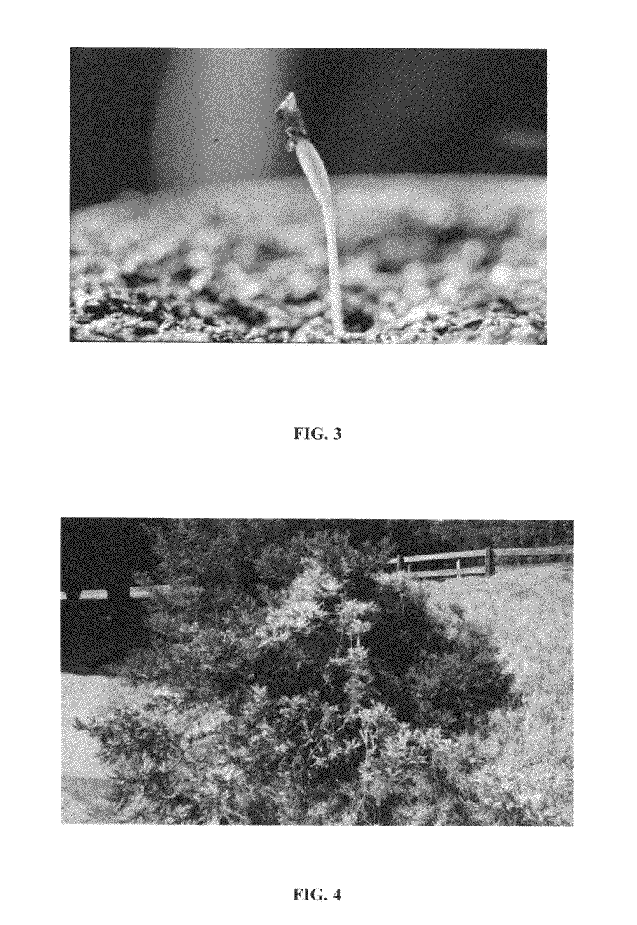

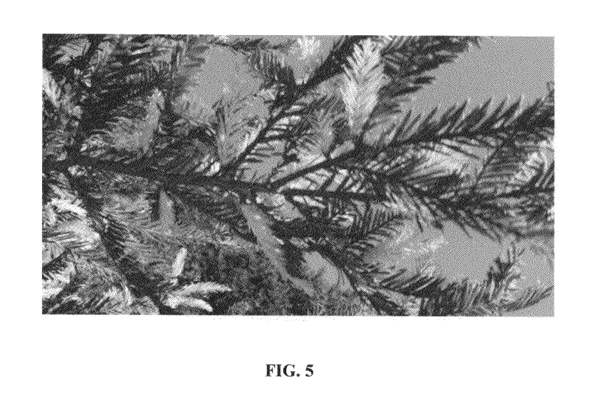

### DETAILED BOTANICAL DESCRIPTION

The following is a detailed description of the new variety ‘Axillares Albus’. Data was collected from a plant approximately 14 months old, from Leaf Cutting Number 2, which was propagated asexually from the present invention. The plant used for a botanical description is presently growing in a greenhouse in Volcano, Calif. The growing conditions approximate those generally used in commercial practice. Color readings were observed indoors with natural lighting diffused through greenhouse panes. The color terms used are of ordinary dictionary significance. Chimeric expression among the propagated plants leads to a variation in color and, therefore, no particular claim to a color's hue, saturation, or intensity is made.

TABLE 2
VARIETY DESCRIPTION

| | |
|---|---|
| Classification: | |
| | Family: Cupressaceae |
| | Botanical: Sequoia sempervirens |
| | Common: Coast Redwood |

| | |
|---|---|
| Parentage: | |
| | Parent One: Unnamed Albino Sequoia sempervirens (neither patented, |
| | nor commercially available) |
| | Parent Two: Unnamed Green Sequoia sempervirens (neither patented, |
| | nor commercially available) |
| Propagation: | |
| | Vegetatively via leaf and stem cuttings |
| Plant: | |
| | Ploidy: Hexaploid |
| | Height, unpruned (m): 84.5 cm (measured at 14 months); potential mature |
| | height of 18 to 36 m |
| | Vigor: Strong |
| | Shape: Pyramidal |
| | Growth rate: Moderate to fast |
| | Growth habit: Horizontal to pendulous |
| | Canopy width (m): 46.0 cm |
| | Canopy height (m): 62.2 cm |
| | Crown shape: Pendulous and pyramidal |
| Trunk and Branchlets: | |
| | Trunk texture: Smooth as cuttings, emerging to fibrous at approximately |
| | four years |
| | Trunk diameter (cm): 1.0 cm taken at 1.0 cm above the ground |
| | Branchlet length (m): 38.1 cm measured from stem |
| | Branchlet texture: Smooth and waxy |
| | Branchlet color: Variable due to chimeric expression, including: green, |
| | white, chimeric, or non-chimeric variegated |
| | Branchlet arrangement: Alternate |
| | Crotch angle from main trunk: |

Green branches: Horizontal range from 0° to 45° and weeping range
from 315° to 360°
Albino, chimeric, or non-chimeric variegated branches:
Approximately 315° to 360° degree

| | |
|---|---|
| Burl: Non observed | |
| Scion/rootstock: | |
| | Circumference (leaf cuttings): 3.0 cm |
| | Height at which measurement taken: 10.6 cm at time of planting and 53.3 |
| | cm for leaf cuttings |
| | Suckering: None observed |
| Leaves: | |
| | Arrangement: Flat needles in alternating patterns |
| | Texture: Glabrous (smooth) |
| | Type: Simple |
| | Shape: Needle-like |
| | Cross section: Concave 1.0 mm |
| | Leaf needle length (mm): 1.0 to 3.0 cm |
| | Leaf needle width (mm): 2.0 to 3.0 cm |
| | Surface: |

a .Upper surface texture: Glabrous (smooth, waxy)
b. Surface color (upper and lower): Green, white, chimeric,
or non-chimeric variegated.

Petiole:

a .Shape: Round and scale-like
b. Color: Green, white, chimeric, or non-chimeric variegated
e. Thorns (spines): Absent

Cones: None observed
Flowers: None observed

Bud:

a. Shape: Scaly
b. Color:

Apical meristem: Chimeric
Lateral buds, adventitious buds, and dorsoventral axillary
buds on the abaxial side of the ramus: Green or white
Petal: None observed

Reproductive Organs: None observed
Fruit: None observed
Best mode growing conditions:

Soil conditions: Deep, well-drained loam and clay-loam soil
Water use/drought tolerance: Require regular watering when young;
however, once established, trees are mildly drought tolerant with optimal
growing conditions including an annual rainfall exceeding 102 cm per
year
Temperature: Best grown in cool climates ranging from 50° F to 80° F
with frost-free winters
Fertilization:

a. Propagation: Potting soil with slow release fertilizer analysis
of 21-7-14
b. Maintenance: Slow release fertilizer

Resistance to disease: Low susceptibility to disease due to tannin content; however,
may be subject to Botryosphaeria sp. canker if under stress conditions (for example, drought)

### COMPARISON TO SIMILAR VARIETIES

Parent One is a non-chimeric variegated albino Sequoia sempervirens demonstrating ninety-five percent albinism with approximately five percent non-chimeric variegation on the new growth and a hedge-bush-like growth habit. Unlike Parent One, ‘Axillares Albus’ displays both chimeric and non-chimeric variegated growth. Specifically, ‘Axillares Albus’ demonstrates stable albino growth from inside the apical meristem and the lateral buds, adventitious buds, accessory buds, and dorsoventral axillary buds on the abaxial side of the ramus. ‘Axillares Albus’ also demonstrates horizontal to weeping-like chimeric branches that are either albino, periclinal, mericlinal, or sectorial.

Parent Two is a standard green Sequoia sempervirens exhibiting characters typical of redwoods grown locally in Santa Cruz, Calif. Parent Two is an old growth, and slow-growing tree without any variegation or chimeric growth. In contrast, ‘Axillares Albus’ grows at a moderate to fast rate, and displays a range of coloration through the chimeric and non-chimeric variegated growth.

The commercially available Sequoia sempervirens named ‘Aptos blue’ demonstrates blue-green foliage and an upright habit with small weeping side branches; whereas, ‘Axillares Albus’ foliage ranges in color from green, albino, chimeric, and non-chimeric variegated and has horizontal to weeping branches.

We claim:

1. A new and distinct variety of periclinal chimeric albino Sequoia sempervirens having the characteristics substantially as described and illustrated herein.
